(19) United States
(12) Patent Application Publication
(10) Pub. No.: US 2025/0308643 A1
(43) Pub. Date: Oct. 2, 2025

(54) SYSTEMS AND METHODS FOR PREDICTED CLASSIFICATION OF SPECIALTY MEDICATIONS BASED ON EXTRACTED PREDICTOR VARIABLES

(71) Applicant: UpToDate, Inc., Waltham, MA (US)

(72) Inventors: Stephen Marc Stout, Ann Arbor, MI (US); Allison Combs, Northville, MI (US); Orinda McIntire, Greeley, CO (US); Jason Coles, Arlington, MA (US)

(21) Appl. No.: 18/621,944

(22) Filed: Mar. 29, 2024

(51) Int. Cl. G16C 20/70; G16H 20/10

(52) U.S. Cl. CPC G16C 20/70; G16H 20/10

(57) ABSTRACT

Systems and methods for automatically determining and indicating specialty medications are disclosed. In some embodiments, a disclosed method includes: obtaining a request from a user for specialty determination of a medication; extracting, based on predictor variables of a machine learning model, relevant data of the medication from at least one database; computing, using the machine learning model, a probability score for the medication based on the relevant data, wherein the probability score indicates a probability that the medication will be determined as a specialty medication; generating, based on the probability score, a specialty indicator indicating whether the medication will be determined as a specialty medication; and transmitting at least one of the specialty indicator or the probability score to the user.

### TECHNICAL FIELD

This application relates generally to machine learning, more particularly, to systems and methods for predicting specialty medication classifications using machine learning.

### BACKGROUND

Health plan members are increasingly prescribed specialty medications (e.g. specialty drugs, biosimilars, cell and gene therapies) for chronic and/or rare diseases. It is increasingly hard to manage medication costs or make coverage decisions with a lack of standard definition of specialty and emerging therapies, and supporting drug data or attributes. Different coverage and policies for medical and pharmacy benefits may increase inconsistency risk. Duplication of efforts that yield variable decisions in different systems can increase risk and costs as well. Currently, users within each organization conduct individual research and monitoring over time and maintain separate systems of record. The information generated in such manner may be biased, incomplete, scattered, referential, and/or change over time. Further, current cost management requires clinical guidance on therapy options, making it hard to stay current on evolving evidence based medicine on corresponding therapies, and is very time consuming for payer clinical personnel.

Specialty medication classifications impact therapeutic and coverage decisions, and predictions of these classifications can help payors and health systems plan for future formulary and coverage decisions. For example, payers need to set policy and formulary that efficiently gets the right health plan members access to the right medications. Current systems are not capable of consistently and accurately providing predicted classifications for use in such decision making processes.

### SUMMARY

The embodiments described herein are directed to systems and methods for generating a predicted classification of specialty medications based on extracted predictor variables.

In various embodiments, a system including a non-transitory memory configured to store instructions thereon and at least one processor is disclosed. The at least one processor is operatively coupled to the non-transitory memory and configured to read the instructions to: obtain a request from a user for specialty determination of a medication; extract, based on predictor variables of a machine learning model, relevant data of the medication from at least one database; compute, using the machine learning model, a probability score for the medication based on the relevant data, wherein the probability score indicates a probability that the medication will be determined as a specialty medication; generate, based on the probability score, a specialty indicator indicating whether the medication will be determined as a specialty medication; and transmit the specialty indicator, or probability score, or both to the user.

In various embodiments, a computer-implementable method is disclosed. The computer-implementable method includes: obtaining a request from a user for specialty determination of a medication; extracting, based on predictor variables of a machine learning model, relevant data of the medication from at least one database; computing, using the machine learning model, a probability score for the medication based on the relevant data, wherein the probability score indicates a probability that the medication will be determined as a specialty medication; generating, based on the probability score, a specialty indicator indicating whether the medication will be determined as a specialty medication; and transmitting the specialty indicator, or probability score, or both to the user.

In various embodiments, a non-transitory computer readable medium having instructions stored thereon is disclosed. The instructions, when executed by at least one processor, cause at least one device to perform operations including: obtaining a request from a user for specialty determination of a medication; extracting, based on predictor variables of a machine learning model, relevant data of the medication from at least one database; computing, using the machine learning model, a probability score for the medication based on the relevant data, wherein the probability score indicates a probability that the medication will be determined as a specialty medication; generating, based on the probability score, a specialty indicator indicating whether the medication will be determined as a specialty medication; and transmitting the specialty indicator, or probability score, or both to the user.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 is a network environment configured for automatically determining and indicating specialty medications, in accordance with some embodiments of the present teaching;

FIG. 2 shows a block diagram of a specialty indication computing device, in accordance with some embodiments of the present teaching;

FIG. 3 shows a block diagram illustrating various portions of a system for automatically determining and indicating specialty medications, in accordance with some embodiments of the present teaching;

FIG. 4 shows an exemplary architecture for automatically determining and indicating specialty medications, in accordance with some embodiments of the present teaching;

FIG. 5 shows an exemplary result of specialty medication determination, in accordance with some embodiments of the present teaching;

FIG. 6 shows a table listing exemplary candidate predictor variables for modeling of specialty medication determination, in accordance with some embodiments of the present teaching;

FIG. 7 illustrates exemplary data types and sources for modeling of specialty medication determination, in accordance with some embodiments of the present teaching;

FIG. 8 illustrates a stepwise model selection for specialty medication determination, in accordance with some embodiments of the present teaching; and

FIG. 9 is a flowchart illustrating an exemplary method for automatically determining and indicating specialty medications, in accordance with some embodiments of the present teaching.

### DETAILED DESCRIPTION

This description of the exemplary embodiments is intended to be read in connection with the accompanying drawings, which are to be considered part of the entire written description. Terms concerning data connections, coupling and the like, such as “connected” and “interconnected,” and/or “in signal communication with” refer to a relationship wherein systems or elements are electrically and/or wirelessly connected to one another either directly or indirectly through intervening systems, as well as both moveable or rigid attachments or relationships, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages or alternative embodiments herein can be assigned to the other claimed objects and vice versa. In other words, claims for the systems can be improved with features described or claimed in the context of the methods. In this case, the functional features of the method are embodied by objective units of the systems.

Specialty medication classifications may be used when determining medical policy, benefit designs, formularies, utilization management, etc. In this disclosure, “specialty medication(s)” or “medication(s)” or “drug(s)” include but not limited to: specialty, specialty drug(s), biosimilar(s), cell therapy/therapies, gene therapy/therapies, cell and gene therapy/therapies, pipeline drug(s), pipeline medication(s), or any other emerging or future therapy/therapies. Methods and systems for automatically determining and indicating specialty medications are disclosed herein, which provide an unbiased clinically-focused content solution designed to power specialty and emerging therapy management.

In some embodiments, a disclosed system can automatically determine whether a medication will be considered or classified as a specialty medication, based on at least one machine learning model, which is pre-modeled or pre-trained based on various extracted and derived medication data. The system allows users (e.g. commercial payers, consultants, and healthcare analytics software firms) to create smarter, faster, end-to-end specialty drug and emerging therapy management efforts in a scalable, automated way. The system also enables the users to make better strategic business decisions about specialty and emerging therapies.

In some embodiments, the system offers specialty indicators and/or associated probability scores to indicate specialty status of some medications desired by users. The specialty indicators and associated probability scores may be incorporated in solutions that supply additional data (e.g., pipeline drug information, detailed cost of therapy, or other data elements used in a score computation model) to support end user decision-making. This provides unbiased and robust data in a structured and codified way that enables user decisions at scale (e.g. higher volume, machine driven, etc.). The disclosed systems and methods provide an improvement to clinical medication applications by increasing accuracy and decreasing variability in predicted or expected classifications of new drugs or therapies, reducing computing resources required to generate predictions, and decreasing the time required to generate classifications. In addition, the disclosed systems and methods enable improvement to additional computing operations based on improvements in classification of new drugs and therapies provided by the disclosed systems and methods.

Further, the disclosed system can improve contracting efficiency, and reduce risk and associated costs (including both administration cost and benefit cost) of inconsistent policies, processes and programs. In addition, the system can reduce time to determine and implement coverage determinations, reduce risk of adverse drug events, reduce clinical variability and associated costs, and reduce provider friction through well-researched requirements based on evidence based medicine (EBM).

The disclosed system is an automatic specialty determination system that can provide insight data to health care users, and free up their time to focus on clinical program development and interventions. The system can also reduce clinical variability and associated costs through application of standardized EBM drug/therapy attributes and indications.

Furthermore, in the following, various embodiments are described with respect to systems and methods for automatically determining and indicating specialty medications are disclosed. In some embodiments, a disclosed method includes: obtaining a request from a user for specialty determination of a medication; extracting, based on predictor variables of a machine learning model, relevant data of the medication from at least one database; computing, using the machine learning model, a probability score for the medication based on the relevant data, wherein the probability score indicates a probability that the medication will be determined as a specialty medication; generating, based on the probability score, a specialty indicator indicating whether the medication will be determined as a specialty medication; and transmitting the specialty indicator, the probability score or both to the user.

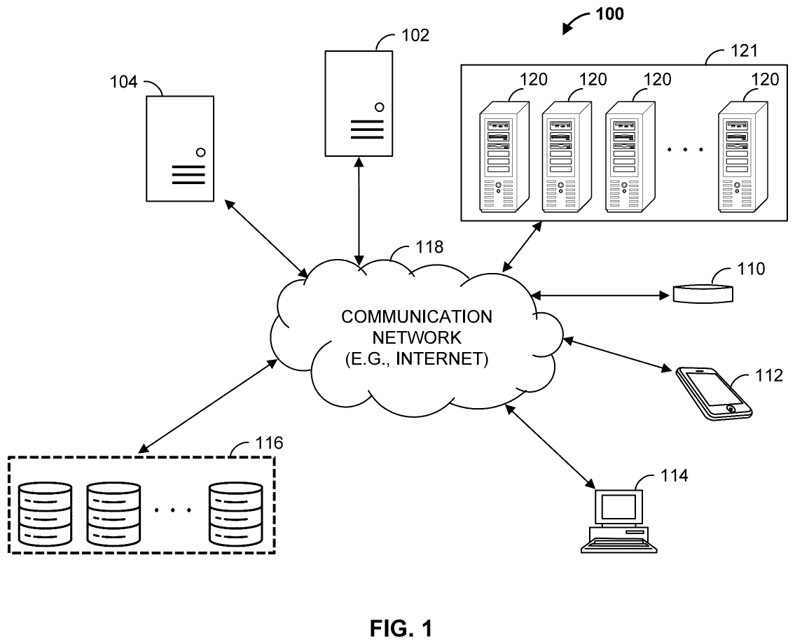

Turning to the drawings, FIG. 1 is a network environment 100 configured for automatically determining and indicating specialty medications, in accordance with some embodiments of the present teaching. The network environment 100 includes a plurality of devices or systems configured to communicate over one or more network channels, illustrated as a network cloud 118. For example, in various embodiments, the network environment 100 can include, but not limited to, a specialty indication computing device 102, a server 104 (e.g., a web server or an application server), a cloud-based engine 121 including one or more processing devices 120, database(s) 116, and one or more user computing devices 110, 112, 114 operatively coupled over the network 118. The specialty indication computing device 102, the server 104, the processing device(s) 120, and the multiple user computing devices 110, 112, 114 can each be any suitable computing device that includes any hardware or hardware and software combination for processing and handling information. For example, each can include one or more processors, one or more field-programmable gate arrays (FPGAs), one or more application-specific integrated circuits (ASICs), one or more state machines, digital circuitry, or any other suitable circuitry. In addition, each can transmit and receive data over the communication network 118.

In some examples, each of the specialty indication computing device 102 and the processing device(s) 120 can be a computer, a workstation, a laptop, a server such as a cloud-based server, or any other suitable device. In some examples, each of the processing devices 120 is a server that includes one or more processing units, such as one or more graphical processing units (GPUs), one or more central processing units (CPUs), and/or one or more processing cores. Each processing device 120 may, in some examples, execute one or more virtual machines. In some examples, processing resources (e.g., capabilities) of the one or more processing devices 120 are offered as a cloud-based service (e.g., cloud computing). For example, the cloud-based engine 121 may offer computing and storage resources of the one or more processing devices 120 to the specialty indication computing device 102.

In some examples, each of the multiple user computing devices 110, 112, 114 can be a cellular phone, a smart phone, a tablet, a personal assistant device, a voice assistant device, a digital assistant, a laptop, a computer, or any other suitable device. In some examples, the server 104 hosts one or more websites or applications. In some examples, the specialty indication computing device 102, the processing devices 120, and/or the server 104 are operated by a data service provider, and the multiple user computing devices 110, 112, 114 are operated by customers of the service or application provided by the data service provider. In some examples, the processing devices 120 are operated by a third party (e.g., a cloud-computing provider).

Although FIG. 1 illustrates three user computing devices 110, 112, 114, the network environment 100 can include any number of user computing devices 110, 112, 114. Similarly, the network environment 100 can include any number of the specialty indication computing devices 102, the processing devices 120, the servers 104, and the databases 116.

The communication network 118 can be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 118 can provide access to, for example, the Internet.

In some embodiments, each of the first user computing device 110, the second user computing device 112, and the Nth user computing device 114 may communicate with the server 104 over the communication network 118. For example, each of the multiple user computing devices 110, 112, 114 may be operable to view, access, and interact with a website or API hosted by the server 104. The server 104 may capture user session data related to a customer's activity (e.g., interactions) on the website or API.

In some examples, a customer may operate one of the user computing devices 110, 112, 114 to access the website (or API) hosted by the server 104. The customer may view services provided on the website, and may click on content items and some notifications, alerts, or other tailored messaging, for example. The website may capture these activities as user session data, and transmit the user session data to the specialty indication computing device 102 over the communication network 118. The website may also allow the customer to add one or more of the services to submit an online inquiry or request for more information. In some examples, the server 104 transmits purchase data identifying services the customer has purchased from the website to the specialty indication computing device 102.

In some examples, the server 104 may transmit a specialty determination request to the specialty indication computing device 102. The specialty determination request may be sent together with conditions and/or queries related to a medication provided by a user (e.g., via API hosted by the data service provider), or a standalone specialty determination request provided by a processing unit in response to the user's action on a website, e.g. clicking a button on the website, submitting a request on the website, etc.

In some examples, upon receiving the specialty determination request, the specialty indication computing device 102 may extract or generate relevant data for the medication from one or more database(s) 116, based on predetermined predictor variables. Based on the relevant data, the specialty indication computing device 102 can compute a probability score indicating a probability that the medication will be considered as a specialty medication. The specialty indication computing device 102 can then generate, based on the probability score, a specialty indicator indicating whether the medication will be considered as a specialty medication, and provide specialty indication data including the specialty indicator and/or the probability score to the user for making decisions about formulary or medical policy.

In some examples, the specialty indication computing device 102 may execute one or more models (e.g., programs or algorithms), such as a machine learning model, deep learning model, statistical model, etc., to generate the specialty indication data. The specialty indication computing device 102 may transmit the specialty indication data to the server 104 over the communication network 118, and the server 104 may display the specialty indication data on the website or via API to users (e.g. commercial payers, consultants, healthcare analytics software firms) who are interested in these data.

In some embodiments, the specialty indication computing device 102 is further operable to communicate with the databases 116 over the communication network 118. For example, the specialty indication computing device 102 can store data to, and read data from, the databases 116. The databases 116 can be a remote storage device, such as a cloud-based server, a disk (e.g., a hard disk), a memory device on another application server, a networked computer, or any other suitable remote storage. Although shown remote to the specialty indication computing device 102, in some examples, the databases 116 can be a local storage device, such as a hard drive, a non-volatile memory, or a USB stick. The specialty indication computing device 102 may store data received from the server 104 in the databases 116. The specialty indication computing device 102 may also store the specialty indication data in the databases 116.

In some examples, the specialty indication computing device 102 generates modeling and/or training data for a plurality of models (e.g., machine learning models, deep learning models, statistical models, algorithms, etc.) based on: e.g. user data, historical medication attribute data, historical specialty indication data, historical user feedback data, etc. The specialty indication computing device 102 develops the models based on their corresponding modeling and/or training data, and stores the models in a database, such as in the databases 116 (e.g., a cloud storage).

The models, when executed by the specialty indication computing device 102, allow the specialty indication computing device 102 to generate specialty indication data based on corresponding datasets. For example, the specialty indication computing device 102 may obtain the models from the databases 116. The specialty indication computing device 102 may receive, in real-time from the server 104, a specialty determination request identifying a request from a user for specialty determination of a medication. In response to receiving the request, the specialty indication computing device 102 may execute the models (and/or retrieve previously executed results) to generate specialty indication data for the medication to be displayed to the user.

In some examples, the specialty indication computing device 102 assigns the models (or parts thereof) for execution to one or more processing devices 120. For example, each model may be assigned to a virtual machine hosted by a processing device 120. The virtual machine may cause the models or parts thereof to execute on one or more processing units such as GPUs. In some examples, the virtual machines assign each model (or part thereof) among a plurality of processing units. Based on the output of the models, the specialty indication computing device 102 may generate specialty indication data to be displayed or delivered to a user.

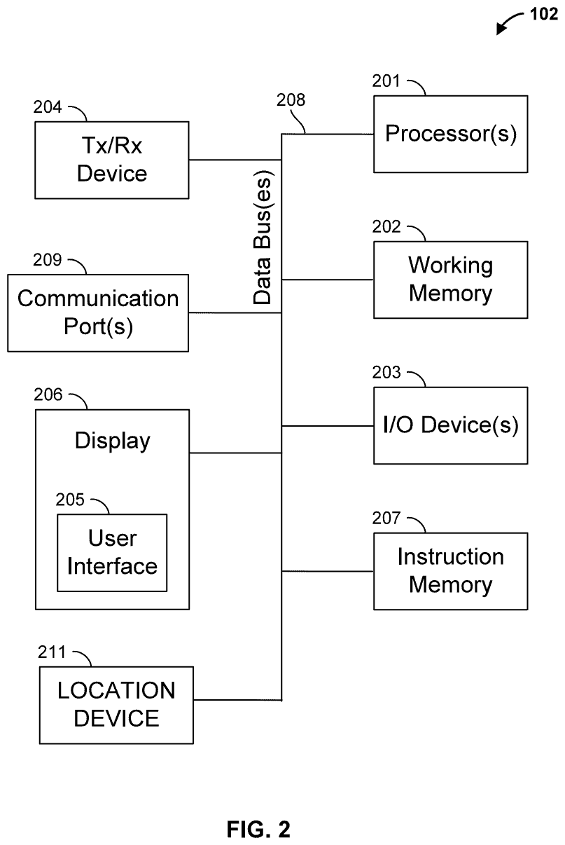

FIG. 2 illustrates a block diagram of a specialty indication computing device, e.g. the specialty indication computing device 102 of FIG. 1, in accordance with some embodiments of the present teaching. In some embodiments, each of the specialty indication computing device 102, the server 104, the multiple user computing devices 110, 112, 114, and the one or more processing devices 120 in FIG. 1 may include the features shown in FIG. 2. Although FIG. 2 is described with respect to certain components shown therein, it will be appreciated that the elements of the specialty indication computing device 102 can be combined, omitted, and/or replicated. In addition, it will be appreciated that additional elements other than those illustrated in FIG. 2 can be added to the specialty indication computing device 102.

As shown in FIG. 2, the specialty indication computing device 102 can include one or more processors 201, an instruction memory 207, a working memory 202, one or more input/output devices 203, one or more communication ports 209, a transceiver 204, a display 206 with a user interface 205, and an optional location device 211, all operatively coupled to one or more data buses 208. The data buses 208 allow for communication among the various components. The data buses 208 can include wired, or wireless, communication channels.

The one or more processors 201 can include any processing circuitry operable to control operations of the specialty indication computing device 102. In some embodiments, the one or more processors 201 include one or more distinct processors, each having one or more cores (e.g., processing circuits). Each of the distinct processors can have the same or different structure. The one or more processors 201 can include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 201 may also be implemented by a controller, a microcontroller, an application specific integrated circuit (ASIC), a field programmable gate array (FPGA), a programmable logic device (PLD), etc.

In some embodiments, the one or more processors 201 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 207 can store instructions that can be accessed (e.g., read) and executed by at least one of the one or more processors 201. For example, the instruction memory 207 can be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 201 can be configured to perform a certain function or operation by executing code, stored on the instruction memory 207, embodying the function or operation. For example, the one or more processors 201 can be configured to execute code stored in the instruction memory 207 to perform one or more of any function, method, or operation disclosed herein.

Additionally, the one or more processors 201 can store data to, and read data from, the working memory 202. For example, the one or more processors 201 can store a working set of instructions to the working memory 202, such as instructions loaded from the instruction memory 207. The one or more processors 201 can also use the working memory 202 to store dynamic data created during one or more operations. The working memory 202 can include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 207 and working memory 202, it will be appreciated that the specialty indication computing device 102 can include a single memory unit configured to operate as both instruction memory and working memory. Further, although embodiments are discussed herein including non-volatile memory, it will be appreciated that the specialty indication computing device 102 can include volatile memory components in addition to at least one non-volatile memory component.

In some embodiments, the instruction memory 207 and/or the working memory 202 includes an instruction set, in the form of a file for executing various methods, e.g. any method as described herein. The instruction set can be stored in any acceptable form of machine-readable instructions, including source code or various appropriate programming languages. Some examples of programming languages that can be used to store the instruction set include, but are not limited to: Java, JavaScript, C, C++, C#, Python, Objective-C, Visual Basic, .NET, HTML, CSS, SQL, NOSQL, Rust, Perl, etc. In some embodiments a compiler or interpreter is configured to convert the instruction set into machine executable code for execution by the one or more processors 201.

The input-output devices 203 can include any suitable device that allows for data input or output. For example, the input-output devices 203 can include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 204 and/or the communication port(s) 209 allow for communication with a network, such as the communication network 118 of FIG. 1. For example, if the communication network 118 of FIG. 1 is a cellular network, the transceiver 204 is configured to allow communications with the cellular network. In some embodiments, the transceiver 204 is selected based on the type of the communication network 118 the specialty indication computing device 102 will be operating in. The one or more processors 201 are operable to receive data from, or send data to, a network, such as the communication network 118 of FIG. 1, via the transceiver 204.

The communication port(s) 209 may include any suitable hardware, software, and/or combination of hardware and software that is capable of coupling the specialty indication computing device 102 to one or more networks and/or additional devices. The communication port(s) 209 can be arranged to operate with any suitable technique for controlling information signals using a desired set of communications protocols, services, or operating procedures. The communication port(s) 209 can include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 209 allows for the programming of executable instructions in the instruction memory 207. In some embodiments, the communication port(s) 209 allow for the transfer (e.g., uploading or downloading) of data, such as machine learning model training data.

In some embodiments, the communication port(s) 209 are configured to couple the specialty indication computing device 102 to a network. The network can include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments can include in-body communications, various devices, and various modes of communications such as wireless communications, wired communications, and combinations of the same.

In some embodiments, the transceiver 204 and/or the communication port(s) 209 are configured to utilize one or more communication protocols. Examples of wired protocols can include, but are not limited to, Universal Serial Bus (USB) communication, RS-232, RS-422, RS-423, RS-485 serial protocols, Fire Wire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols can include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1×RTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBee, etc.

The display 206 can be any suitable display, and may display the user interface 205. For example, the user interfaces 205 can enable user interaction with the specialty indication computing device 102 and/or the server 104. For example, the user interface 205 can be a user interface for an application of a network environment operator that allows a customer to view and interact with the operator's website. In some embodiments, a user can interact with the user interface 205 by engaging the input-output devices 203. In some embodiments, the display 206 can be a touchscreen, where the user interface 205 is displayed on the touchscreen.

The display 206 can include a screen such as, for example, a Liquid Crystal Display (LCD) screen, a light-emitting diode (LED) screen, an organic LED (OLED) screen, a movable display, a projection, etc. In some embodiments, the display 206 can include a coder/decoder, also known as Codecs, to convert digital media data into analog signals. For example, the visual peripheral output device can include video Codecs, audio Codecs, or any other suitable type of Codec.

The optional location device 211 may be communicatively coupled to a location network and operable to receive position data from the location network. For example, in some embodiments, the location device 211 includes a GPS device configured to receive position data identifying a latitude and longitude from one or more satellites of a GPS constellation. As another example, in some embodiments, the location device 211 is a cellular device configured to receive location data from one or more localized cellular towers. Based on the position data, the specialty indication computing device 102 may determine a local geographical area (e.g., town, city, state, etc.) of its position.

In some embodiments, the specialty indication computing device 102 is configured to implement one or more modules or engines, each of which is constructed, programmed, configured, or otherwise adapted, to autonomously carry out a function or set of functions. A module/engine can include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine can also be implemented as a combination of the two, with certain functions facilitated by hardware alone, and other functions facilitated by a combination of hardware and software. In certain implementations, at least a portion, and in some cases, all, of a module/engine can be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine can be realized in a variety of physically realizable configurations, and should generally not be limited to any particular implementation exemplified herein, unless such limitations are expressly called out. In addition, a module/engine can itself be composed of more than one sub-modules or sub-engines, each of which can be regarded as a module/engine in its own right. Moreover, in the embodiments described herein, each of the various modules/engines corresponds to a defined autonomous functionality; however, it should be understood that in other contemplated embodiments, each functionality can be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

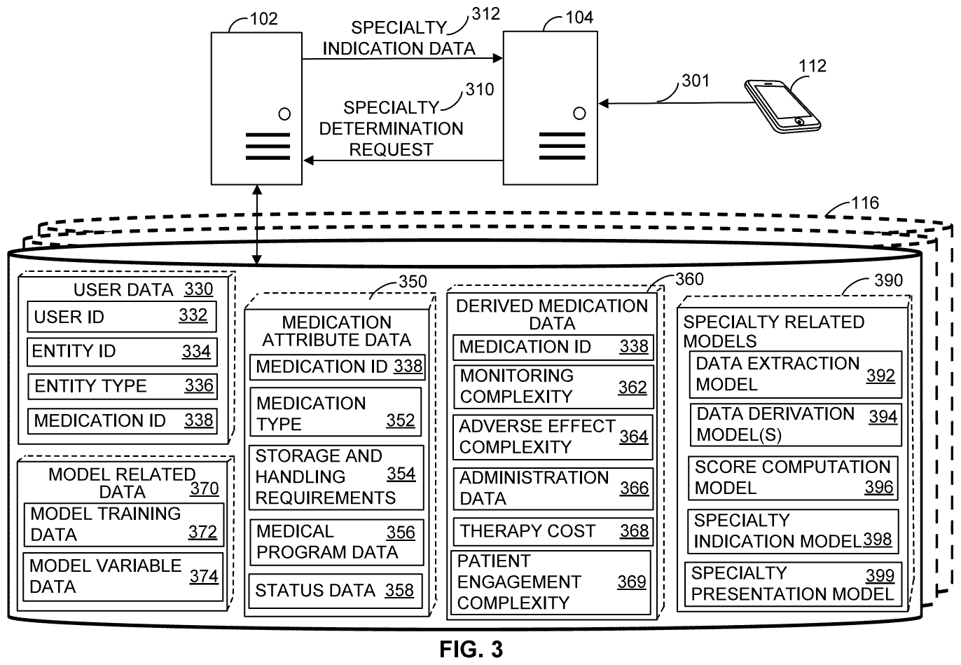

FIG. 3 is a block diagram illustrating various portions of a system for automatically determining and indicating specialty medications, e.g. the system shown in the network environment 100 of FIG. 1, in accordance with some embodiments of the present teaching. As discussed above, the specialty indication computing device 102 may receive user session data from the server 104, and store the user session data in the databases 116.

The specialty indication computing device 102 may parse the user session data to generate user data 330. In this example, the user data 330 may include, for each user of the server 104, one or more of: a user identity (ID) 332 identifying the user, an entity ID 334 identifying an entity associated with the user, an entity type 336 identifying a type of the entity or the user (e.g. commercial payers, pharmacy benefit managers, consultants, healthcare analytics software firms, etc.), and a medication ID 338 identifying medication(s) queried by or associated with the user. The specialty indication computing device 102 and/or the server 104 may store the user data 330 in the databases 116.

The databases 116 may also store medication attribute data 350, which may identify data of medication attributes for any given medication. The medication attribute data 350 may include: the medication ID 338 identifying a medication, medication type 352 identifying a type of the medication (e.g. whether the medication is biologic or non-biologic), storage and handling requirements 354 identifying special requirements for storing and handling the medication, medical program data 356 identifying whether the medication is included in a medical program (e.g. a government-based program about risk evaluation and mitigation strategies), and status data 358 identifying a medication status (e.g. orphan drug status) associated with the medication.

The databases 116 may also store derived medication data 360, which may identify derived medication data for any given medication. The derived medication data 360 may include: the medication ID 338 identifying a medication, monitoring complexity 362 identifying a code or degree of monitoring needed for a patient taking the medication, adverse effect complexity 364 identifying a code or degree of adverse effect resulting from the medication, administration data 366 identifying any administration needed by a health care professional for the medication, therapy cost 368 identifying cost of therapy for the medication, including costs related to dosing, course of therapy, and waste estimates, and patient engagement complexity 369 identifying a code or degree of patient engagement needed for the medication to be successful.

The databases 116 may also store the specialty related models 390 identifying and characterizing one or more models for automatically determining and indicating specialty medications. For example, the specialty related models 390 may include a data extraction model 392, data derivation model(s) 394, a score computation model 396, a specialty indication model 398, and a specialty presentation model 399.

The data extraction model 392 may be used to extract medication related data from a database, e.g. an external database. In some embodiments, the data extraction model 392 is based on an optical character recognition (OCR) and a natural language processing (NLP). For example, after OCR is applied to recognize textual information of a document, the NLP can be applied to determine boundaries of the textual data meeting predetermined conditions or queries. In some examples, the medication related data extracted by the data extraction model 392 is stored as the medication attribute data 350 in the database(s) 116. In some examples, the medication related data extracted by the data extraction model 392 is used to generate the derived medication data 360 in the database(s) 116.

The data derivation model(s) 394 may be used to derive medication data for any given medication and store as the derived medication data 360 in the database(s) 116. For example, the data derivation model(s) 394 can be used to derive the monitoring complexity 362, the adverse effect complexity 364, and/or other derived medication data 360, for a medication based on medication related data extracted for the medication by the data extraction model 392, and based on some predetermined rules or criteria. The rules or criteria may be computed based on inputs from experts, historical derived medication data for similar medications, historical specialty determination data, and/or historical user feedbacks.

The score computation model 396 may be used to compute a probability score indicating a probability that the medication will be determined as a specialty medication. In some examples, the score computation model 396 is used to compute the probability based on the medication attribute data 350 and/or the derived medication data 360 related to the medication. The score computation model 396 may be updated as the medication attribute data 350 and/or the derived medication data 360 changes, for example through addition of new data attributes, deletion of existing data attributes, or adjusting model parameter values.

The specialty indication model 398 may be used to generate specialty indication data for a medication. In some examples, the specialty indication model 398 is used to generate specialty indication data based on the probability score computed by the score computation model 396. For example, the specialty indication model 398 may be used to generate a specialty indicator indicating whether the medication will be determined as a specialty medication, by comparing the probability score with a threshold. The threshold can be adaptively updated based on the user data 330 and/or some user feedback data. In some examples, the specialty indication data may include: the specialty indicator, the probability score, and the associated medication ID.

The specialty presentation model 399 may be used to determine a presentation style for the generated specialty indication data. The specialty presentation model 399 may indicate: a type of user interface for displaying the specialty indication data, content to be displayed together with the specialty indication data in the user interface, layout of different content in the user interface, a document format for the specialty indication data, etc.

The databases 116 may also store model related data 370 for one or more of the data extraction model 392, the data derivation model(s) 394, the score computation model 396, the specialty indication model 398 and the specialty presentation model 399. The model related data 370 may include: modeling and/or model training data 372 identifying modeling and/or training data for development of one or more of these models, and model variable data 374 identifying variables used in one or more of these models for model-based inference. For example, the model variable data 374 for the score computation model 396 may include one or more predication variables from the medication attribute data 350 and the derived medication data 360 to be used by the score computation model 396 for computing the probability score for medications.

In some embodiments, one or more of the data extraction model 392, the data derivation model(s) 394, the score computation model 396, the specialty indication model 398 and the specialty presentation model 399 are machine learning models developed based on some modeling and/or training data, e.g. the model training data 372. In some embodiments, the modeling and/or training data includes labelled data and feedback data. For example, the modeling and/or training data for the score computation model 396 may include labelled specialty status based on historical data for medications, and relevant data (e.g. the medication attribute data 350 and the derived medication data 360) for the medications.

As indicated in FIG. 3, the specialty indication computing device 102 may receive from the server 104 a specialty determination request 310 as a message 301 is sent from the user device 112 to the server 104. The specialty determination request 310 may be associated with an entity type or a customer configuration of a user using the user device 112. In response to the specialty determination request 310, the specialty indication computing device 102 generates specialty indication data 312 identifying whether and how likely a medication will be determined as a specialty medication, and transmits the specialty indication data 312 to the server 104.

In some examples, the specialty indication computing device 102 may extract, based on predictor variables of a machine learning model, e.g. the score computation model 396, relevant data of the medication from at least one database, e.g. the database(s) 116. Using the score computation model 396, the specialty indication computing device 102 can compute a probability score for the medication based on the relevant data, the probability score indicating a probability that the medication will be determined as a specialty medication. Based on the probability score, the specialty indication computing device 102 may generate a specialty indicator indicating whether the medication will be determined as a specialty medication, e.g. using the specialty indication model 398. The specialty indication data 312 may include the specialty indicator and/or the probability score associated with the medication.

After receiving the specialty indication data 312, the server 104 may display the specialty indication data 312 to the user via the user device 112, based on an instruction from the specialty indication computing device 102 according to the specialty presentation model 399. The user, for example, may be a payer who will use the specialty indication data 312 to make decisions for a health care coverage, or medication cost management. In some embodiments, the user may be a pharmacy benefit manager, a consultant, a healthcare analytics software firm, or any other user desiring specialty related indication data of medications.

In some embodiments, the specialty indication computing device 102 may assign one or more of the above described operations to a different processing unit or virtual machine hosted by one or more processing devices 120. Further, the specialty indication computing device 102 may obtain the outputs of the these assigned operations from the processing units, and generate the specialty indication data 312 based on the outputs.

In some embodiments, the specialty determination request 310 is generated before or without the server 104 receiving the message 301 from the user device 112. For example, the specialty indication computing device 102 can pre-compute the specialty indication data 312 for any or all medications with medication IDs 338 stored in the databases 116. The specialty indication computing device 102 may either send the specialty indication data 312 to the server 104 or store the specialty indication data 312 in the databases 116. Then if the server 104 receives the message 301 from the user device 112 afterwards, the server 104 can directly provide the specialty indication data 312 (obtained from the specialty indication computing device 102 or retrieved from the databases 116) to the user device 112, without any re-computation.

In some embodiments, the specialty indication data 312 provided to the user device 112 may include more data than what is requested by the user via the message 301. In some examples, the specialty indication data 312 pre-computed by the specialty indication computing device 102 includes a complete dataset in form of a webpage or a document. Then if the data requested by the user is part of the dataset, the entire webpage or the entire document can be provided to the user as a response to the request. In some examples, if the user is asking a specialty indicator for one drug, the specialty indication data 312 pre-computed by the specialty indication computing device 102 and provided to the user may include specialty indicators (and/or associated probability scores) for a list of multiple drugs including the drug requested by the user.

In some embodiments, a user first sends a transaction request for a product (or a selected set of products or services), e.g. via a purchase order. After buying the selected product, the user obtains a license to access the product through a website, database or API, e.g. hosted by the server 104. Then later when the user sends a request for data related to the purchased product, the server 104 can obtain relevant data from the specialty indication computing device 102 or the databases 116 upon validating the license of the user, and provide the relevant data to the user. In some examples, the request is a data processing request or a data computation request, where the specialty indication computing device 102 only computes the relevant data after the user obtains the license and sends the data processing request or data computation request. In other examples, the request is an access request for pre-computed relevant data, where the specialty indication computing device 102 has pre-computed the relevant data for the user to download or access (upon validation of license) before receiving the access request. In some situations, the specialty indication computing device 102 may have pre-computed the relevant data even before the user obtains the license or sends the transaction request. In various embodiments, the message 301 may be associated with a transaction request, a data processing request, a data computation request, or an access request. In various embodiments, the specialty indication data 312 may be relevant data computed after receiving the user request or pre-computed before receiving the user request.

In some embodiments, the specialty indication computing device 102 and the server 104 may be implemented as multiple systems or in cooperation with multiple systems, including e.g. system(s) for the user to make a request, system(s) for the user to download a document, system(s) for the user to view, access or manage a document. In some embodiments, based on a license or contract with some users, the users may be provided the ability to include the specialty indication data 312 in their external reports or as part of their analytic efforts with some third party consultants.

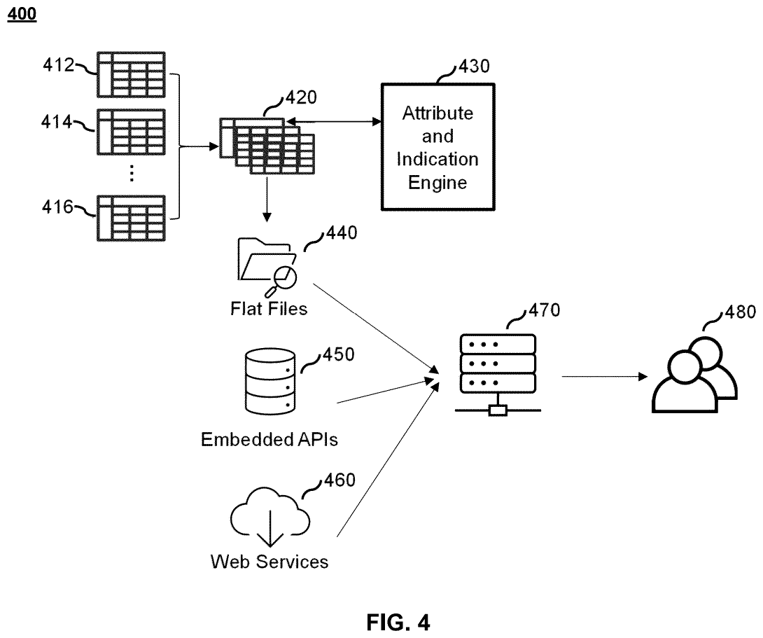

FIG. 4 shows an exemplary architecture 400 for automatically determining and indicating specialty medications, in accordance with some embodiments of the present teaching. In the example shown in FIG. 4, the architecture 400 includes several existing data tables 412, 414, 416, a data staging table 420, an attribute and indication engine 430, flat files 440, embedded application programming interfaces (APIs) 450, web services 460, and a user-accessible database 470. In some examples, one or more of the attribute and indication engine 430, the embedded APIs 450 and the web services 460 are implemented in hardware. In some examples, one or more of the existing data tables 412, 414, 416, the data staging table 420, the attribute and indication engine 430, the flat files 440, the embedded APIs 450, the web services 460, and the user-accessible database 470 are implemented in hardware. In some examples, one or more of the existing data tables 412, 414, 416, the data staging table 420, the attribute and indication engine 430, the flat files 440, the embedded APIs 450, the web services 460, and the user-accessible database 470 are implemented as an executable program maintained in a tangible, non-transitory memory, such as instruction memory 207 of FIG. 2, which may be executed by one or more processors, such as the processor 201 of FIG. 2.

In some examples, the existing data tables 412, 414, 416 are stored in one or more databases, e.g. the database(s) 116, and include various medication data, e.g. data from the medication attribute data 350 and/or the derived medication data 360. In some examples, the attribute and indication engine 430 is implemented as part of the specialty indication computing device 102, and is configured to derive additional medication data and generate specialty indication data. For example, the additional medication data may be derived based on inputs including new manually developed content from medical experts, e.g. clinical content staff. In some embodiments, logic and rules can be set and incorporated into scalable replacement to derive the additional medication data, e.g. based on one of the data derivation model(s) 394. In some embodiments, the attribute and indication engine 430 utilizes the score computation model 396 and the specialty indication model 398 to generate the specialty indication data, based on the medication data in the existing data tables 412, 414, 416 and the derived additional medication data.

In some examples, the data staging table 420 pulls in both the new manual content and derived additional medication data from the attribute and indication engine 430, and brings in appropriate content from the existing data tables 412, 414, 416. The data staging table 420 in one example integrates all data together in a minimal format, but is expandable and flexible for later additions and enhancements. In some embodiments, the specialty indication data is also included in the data staging table 420. In some embodiments, the data staging table 420 may also include additional features like speed to market, availability of content, etc.

In some examples, data content in one of the existing data tables 412, 414, 416 is not in a machine readable format consistent with the data staging table 420. The data staging table 420 would convert that data content into a consistent machine readable format in that case. In some examples, new content generated by the attribute and indication engine 430 is not machine readable. In that case, the data staging table 420 can either convert that new content to a machine readable format or bring in other machine readable content to marry up with the new content generated by the attribute and indication engine 430 into the staging table.

The data staging table 420 may then be used to create flat files 440 for delivering specialty medication data. In some embodiments, the specialty medication data in the data staging table 420 can be delivered with different options, e.g. in flat files 440, via the embedded APIs 450 and/or via web services 460, based on users' requests or requirements.

In some embodiments, the user-accessible database 470 is a database associated with one or more of the flat files 440, the embedded APIs 450 and the web services 460. In some examples, the user-accessible database 470 can provide standardized and codified medication insight data including a specialty indicator, which enables automated decisions across multiple applications by users 480.

The users 480 may include customers like payers, pharmacy benefit managers, consultants, healthcare analytics software firms. In some embodiments, the data provided by the user-accessible database 470 can be automatically added to data universes and applications on the user side, to provide solution accessible to payer medical and pharmacy teams, health plan consultants, analytics, payers, pharmacy benefit managers, etc. In some embodiments, upon buying or obtaining a license, the users 480 can download or retrieve data from the user-accessible database 470, and perform user-side decisions based on the downloaded or retrieved data.

In some embodiments, these solutions can help payers align medical and pharmacy management of specialty drugs and emerging therapies, and reduce provider abrasion. The solutions can also help payers or pharmacy benefit managers to make better decisions on: medical policy, benefit design, formularies, ad clinical programs, utilization management, prior authorization, claims adjudication, appeals, and audits, analytics, medication therapy management, health plan member touchpoints, etc. For consultants and healthcare analytics software firms and others, the solutions can enable their clients to make business decisions about specialty drugs and emerging therapies, and can also help them on: commercialization of new products or programs, competitive positioning, optimization of on-market products or programs, cost analysis, strategy development, etc.

In some embodiments, a disclosed system develops both a specialty indicator (SI) and an SI probability score (SI-PS) as proprietary data elements to help guiding specialty medication formulary decisions. The SI-PS is a probability score generated at a medication level (e.g., at the level of a US Food and Drug Administration National Drug Code) that represents a likelihood the medication will be considered a specialty medication, e.g. for formulary decision making. The SI will be a suggested specialty categorization (e.g. as YES or NO) based on a pre-established SI-PS cutoff threshold.

In some embodiments, the SI and SI-PS are provided to a user via a web service, API or other file application to allowing users to submit drug data and generate their own results, as shown in FIG. 3 and FIG. 4. In some embodiments, the SI and SI-PS are computed without any user interface or cloud component. In some examples, to calculate a group of SI-PS scores and make SI assignments, the system can read in a data report of drug attributes derived from related databases to a working memory, calculate model results for all drugs using some basic linear algebra, and output a modified version of the original data report with SI-PS and SI variables included. The output may then be incorporated in a data system or database. The final SI-PS and SI values will be provided to users as static values. In some examples, the SI-PS model can be incorporated in an editorial system to automatically populate values for any drugs whose requisite information is available.

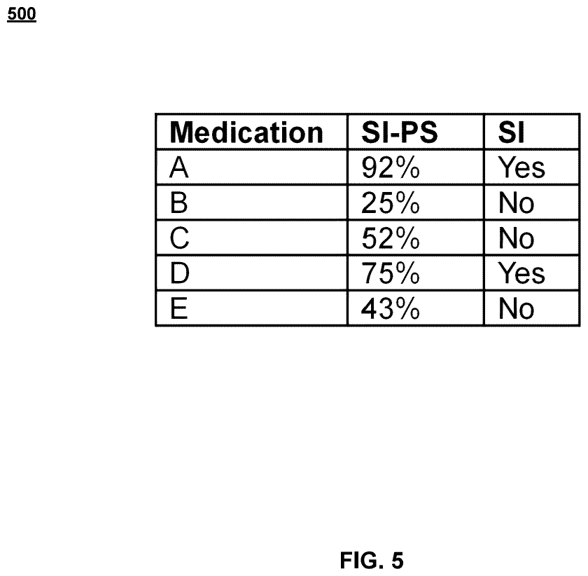

In some embodiments, the SI and SI-PS are created for medications that have been granted regulatory approval, e.g. Federal Drug Administration approval of a NDA, BLA, etc., (called approved medications from here on) as well as for medications that are in later stages of pre-approval development (called pipeline medications from here on). FIG. 5 shows an exemplary table 500 including results of specialty medication determination, in accordance with some embodiments of the present teaching. For a fictional set of approved medications A, B, and C, and pipeline medications D and E, FIG. 5 shows their SI-PS and SI, respectively. As shown in FIG. 5, each SI-PS takes a value as a percentage number, and each SI takes a value of YES or NO. In the example shown in FIG. 5, an SI cutoff threshold of >60% is used to generate the SI. In some embodiments, the SI cutoff value can be changed or updated based on various factors like user data, medication data, user requirements, etc.

In some embodiments, the SI-PS computation model and the cutoff value for SI are periodically recalculated to incorporate new data and optimize performance. In some examples, a sample of medications can be used to create an initial model. The SI can be derived initially by modeling the impact of several proprietary (internally derived or stored) and non-proprietary (externally or publicly available) data elements present in databases (e.g. the database(s) 116) on specialty medication status using logistic regression with maximum likelihood estimation. The specialty medication status may be determined by the clinical content experts and is considered the source of truth for specialty vs. non-specialty status for training or generating the initial model.

In some embodiments, the model is established via stepwise model selection evaluating a plurality of candidate variables, which include but not limited to measures of: indication from medications; medication types (e.g., biologic vs. non-biologic) of the medications; baseline drug attributes and codes (including packaging, therapeutic class, price); specialty drug attributes; cost of therapy for the medications; special storage conditions for the medications; special preparation conditions for the medications; special handling conditions for the medications; requirements for administration by health-care providers; inclusion in a risk evaluation and mitigation program, e.g. government-based Risk Evaluation and Mitigation Strategies (REMS) program; orphan medication status; adverse effect complexity; medication monitoring complexity; patient engagement complexity; pipeline status and clinical attributes where appropriate and available; clinical attributes (including indications, therapeutic alternatives, etc.); clinical level of engagement, route of administration, dispensing requirements, etc.

In some embodiments, the adverse effect complexity for a medication is represented by an adverse effect complexity code indicating a degree of adverse effect resulting from the medication. The adverse effect complexity code may be one of: low, moderate or high, assigned based on an input of an expert or automatically based on at least one predetermined rule or criteria. In some examples, a low adverse effect complexity code means it is unlikely to have an adverse effect when taking the medication, or the adverse effect is simple to manage and not impacting quality of life. In some examples, a high adverse effect complexity code means the adverse effect leads to a hospitalization or serious life threatening complications, and definitely need to be managed and often prevented, or the patients need to take medication specifically to proactively prevent that adverse effect from occurring. In some examples, a moderate adverse effect complexity code means the adverse effect would be somewhere between those of the low and high adverse effect complexity codes.

In some embodiments, the medication monitoring complexity for a medication is represented by a monitoring parameters complexity code indicating a degree of monitoring needed for a patient taking the medication. The monitoring parameters complexity code may be one of: low, moderate or high, assigned based on an input of an expert or automatically based on at least one predetermined rule or criteria. In some examples, a low monitoring parameters complexity code means a patient taking the medication needs to have a small number of lower-risk medical evaluations and tests, e.g. monitoring of their weight and occasional blood analysis. In some examples, a high monitoring parameters complexity code means a patient needs to have intensive monitoring of a large number of parameters and/or monitoring that is highly invasive (e.g., requiring bone marrow aspiration). In some examples, a moderate monitoring parameters complexity code means the monitoring requirement or frequency would be somewhere between those of the low and high monitoring parameters complexity codes.

In some embodiments, the patient engagement complexity is represented by a patient engagement critical to success code indicating a degree of patient engagement needed for the medication to be successful. The patient engagement critical to success code may be one of: low, moderate or high, assigned based on an input of an expert or automatically based on at least one predetermined rule or criteria. In some examples, a low patient engagement critical to success code means a patient taking the medication does not need to perform any engagement or monitoring other than taking the medication, to ensure a successful or expected effect of the medication. In some examples, a high patient engagement critical to success code means a patient taking the medication has to self-perform, or with help of a caregiver, some medical tests, adverse effect monitoring, or taking medication to prevent certain effects, to ensure a successful or expected effect of the medication in the absence of a healthcare professional. In some examples, a moderate patient engagement critical to success code means the engagement requirement for the patient would be somewhere between those of the low and high patient engagement critical to success codes. The patient engagement critical to success code would be a critical factor for payers to consider especially for expensive medications to ensure their effectiveness.

In some examples, a monitoring parameters database is established, as a standalone database or part of the database(s) 116. One or more criteria are set for evaluating medication monitoring complexity of a given medication, including: e.g. how many monitoring parameters are too much, how many monitoring parameters would make the code high, which specific parameters are markers that either need a medical specialist involved, or need a sample drawn from some body fluid, etc. With these set criteria, the system can automatically determine a monitoring parameters complexity code for a given medication without expert input. With criteria similarly set for adverse effect complexity and patient engagement complexity, the system can also automatically determine an adverse effect complexity code and a patient engagement critical to success code for any given medication without expert input.

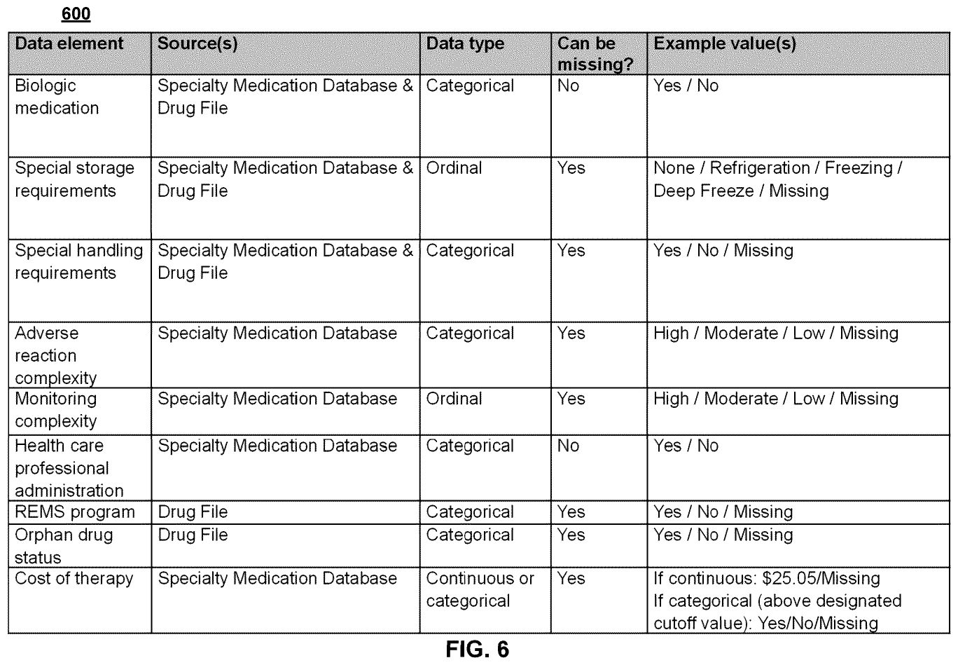

FIG. 6 shows a table 600 listing exemplary candidate predictor variables for modeling of specialty medication determination, in accordance with some embodiments of the present teaching. Some variables such as inclusion in a REMS program are only available for approved medications. That is, some pipeline or non-approved medications may have missing or unknown values. In some embodiments, the SI-PS model can be generated to accommodate a limited range of missing values to account for these situations. For example, the REMS program status may be assigned values of Yes, No, or Missing, as shown in FIG. 6. For example, the monitoring complexity may be assigned values of High, Moderate, Low, or Missing, as shown in FIG. 6.

As shown in FIG. 6, sources and data types for each data element or data variable are listed. In some embodiments, ordinal predictor variables are treated as categorical in the initial modeling and may be consolidated into binary strata (e.g., higher vs. lower). Missing values are expected for some variables (e.g., for characteristics that cannot be assessed for pipeline medications) and can be modeled for some additional variables if data availability is inconsistent. The cost of therapy may be modeled as a continuous variable and/or as a categorical variable to assess which has a stronger association with specialty status and better predictive validity, and one of these will be retained in the final model.

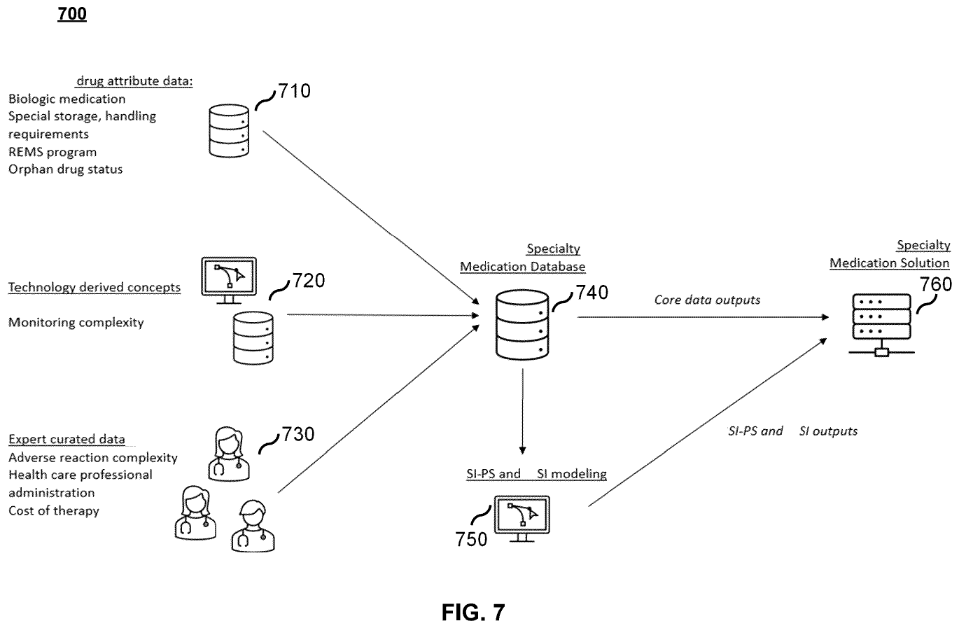

In some examples, a sample of medications (e.g. 500˜5000 medications) including around 10-20% specialty medications are used to establish an initial SI-PS model. FIG. 7 illustrates a framework 700 for modeling of specialty medication determination from exemplary data types and sources, in accordance with some embodiments of the present teaching. In some embodiments, the framework 700 can be carried out by one or more computing devices, such as the server 104 and/or the specialty indication computing device 102 of FIG. 1.

As shown in FIG. 7, the framework 700 includes a specialty medication database 740 that pulls data from a drug attribute database 710, a technology derived database 720, and expert curated data 730. In some embodiments, the drug attribute database 710 includes medication attribute data, e.g. biologic medication, special storage and handling requirements, REMS program, orphan drug status. In various embodiments, the drug attribute database 710 can be standalone database, or part of the database(s) 116 to include the medication attribute data 350.

In some embodiments, the technology derived database 720 includes technology derived data, e.g. monitoring complexity. In some embodiments, the expert curated data 730 may include items such as adverse effect complexity, health care professional administration, and cost of therapy. In some embodiments, one or more elements of the expert curated data 730 are automatically generated as technology derived data in the technology derived database 720. In various embodiments, the technology derived database 720 and the expert curated data 730 can be components of standalone databases, or part of the database(s) 116 to include the derived medication data 360.

During a model development stage of the system, the specialty medication database 740 pulls all data from the drug attribute database 710, the technology derived database 720 and the expert curated data 730, and sends the data to a modeling module 750 for generating an initial model. The initial model can be updated based on additional data or medication attributes to generate an updated SI-PS model.

During an inference stage of the system, the specialty medication database 740 pulls merely relevant data from the drug attribute database 710, the technology derived database 720 and the expert curated data 730 for a given medication, and sends the relevant data to the modeling module 750 for applying the SI-PS model to the relevant data, to generate or predict the SI-PS and SI for the medication based on a cutoff threshold. The relevant data may be determined based on predictor variables selected by the SI-PS model from the data attributes in any or all of the drug attribute database 710, the technology derived database 720 and the expert curated data 730. A final specialty medication solution 760 provided to a user will include both core data outputs from the specialty medication database 740 and SI-PS and SI outputs generated by the SI-PS model from the modeling module 750. The core data outputs may include core information related to a medication queried by the user. The SI-PS and SI outputs may include both the SI-PS indicating a probability that the medication will be determined as a specialty medication, and the SI indicating whether the medication will be determined as a specialty medication. In some embodiments, the final specialty medication solution 760 also includes criteria considerations and/or model related information, for the user to understand how the solution is generated.

In some embodiments, the SI-PS model is a machine learning model developed based on a plurality of candidate variables of a set of medications. For each of the plurality of candidate variables, it is determined whether a relation between the candidate variable and a specialty medication status predetermined for the medications is sufficiently strong, based on a parameter estimate P-value below a predetermined threshold value from logistic regression with maximum likelihood estimation. The specialty medication status may be predetermined by an expert team and/or user feedback. In accordance with a determination that the parameter estimate P-value is lower than the threshold, the candidate variable is incorporated as one of the predictor variables in the machine learning model. In accordance with a determination that the P-value is not lower than the threshold, the candidate variable is excluded from the machine learning model.

In some embodiments, the SI-PS model is updated after receiving final or actual classifications of the corresponding medications, e.g. based on a user feedback, health insurance definition, or drug classification institution. For example, the modeling or training dataset can be updated to incorporate the final classifications, and re-model or re-train the model based on the actual classifications of the medications.

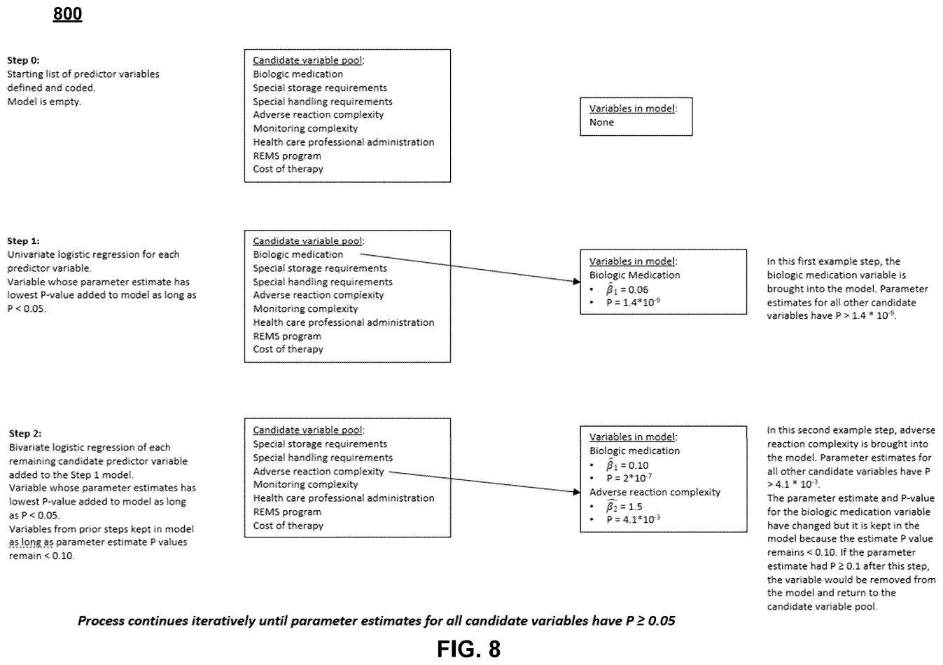

FIG. 8 illustrates a process 800 of a stepwise model selection for generating or training a model for specialty medication determination, in accordance with some embodiments of the present teaching. In some embodiments, the process 800 can be carried out by one or more computing devices, such as the specialty indication computing device 102 of FIG. 1.

As shown in FIG. 8, the process 800 starts from step 0, where a starting list of predictor variables are defined and coded. In this example, the candidate variable pool includes: biologic medication, special storage requirements, special handling requirements, adverse reaction complexity, monitoring complexity, health care professional administration, REMS program, and cost of therapy. At step 0, the model is empty with no variable.

At step 1 of the process 800, a univariate logistic regression is performed for each predictor variable. A variable whose parameter estimate has lowest P-value is added to the model as long as P<0.05. In some examples, the P-value is a probability of finding the current parameter estimate or a more extreme (farther from zero) value if there is no true relation between the variable and specialty medication classification (null hypothesis). In many scientific conventions, if this probability is lower than 5% (P<0.05), the relation is considered statistically significant. In the example shown in FIG. 8, the biologic medication variable is brought into the model at step 1.

Then, at step 2 of the process 800, a bivariate logistic regression is performed including each remaining candidate predictor variable (1 by 1) in the step 1 model. A variable whose parameter estimate has the lowest P-value is added to model as long as P<0.05, where variables from prior steps are kept in the model as long as parameter estimate P values remain less than 0.10. In the example shown in FIG. 8, the adverse reaction complexity is brought into the model at step 2. At step 2, the parameter estimate and P-value for the biologic medication variable have changed, but the biologic medication variable is kept in the model because its estimate P value remains less than 0.10. If its parameter estimate had P≥0.1 after this step, the biologic medication variable would be removed from the model and return to the candidate variable pool.

The process 800 continues iteratively for the remaining candidate predictor variables in the candidate variable pool, until parameter estimates for all remaining candidate variables not yet in the model have P≥0.05, to generate a final model for specialty medication determination.

In some embodiments, the final model has a general form of:

$$P ( SI - PS = 1 ) = \frac{1}{1 + {e}^{- ( {\hat{β}}_{1} * {P}_{1} + {\hat{β}}_{2} * {P}_{2} + {\hat{β}}_{3} * {P}_{3} + … + {\hat{β}}_{0} )}}$$

which is a model for the probability that a medication will be considered a specialty medication (SI-PS=1) based on predictor variables (denoted P₁, P₂, etc.) and parameter estimates (\hat{β} terms) established through the modeling depicted in FIG. 7.

In some embodiments, with exemplary parameter values (\hat{β} terms), the final model has an exemplary specific form of:

$$P ( SI - PS = 1 ) = \frac{1}{1 + {e}^{- 0.76 * MC + 0.44 * {MC}_{M} - 0.98 * AC + 0.4 * {AC}_{M} - 0.3 * Bio - 0.15 * R + 0.21 * {R}_{M} - 1 * SH + 0.65 * {SH}_{M} - 0.000076 * CT + 1.13}}$$

which is an exemplary probability model for SI-PS based on predictor variables monitoring complexity (MC, as simplified binary strata [high vs. not high]), adverse reaction complexity (AC, as binary strata), biologic medication (Bio), REMS requirement (R), special handling requirements (SH), and cost of therapy (CT, as continuous variable). Variables denoted with a subscript M are for missing values. An example medication with high monitoring complexity (MC=1, MC_{M}=0), missing adverse reaction complexity (AC=0, AC_{M}=1), positive biologic status (Bio=1), REMS requirement (R=1, R_{M}=0), special handling requirement (SH=1, SH_{M}=0), and $1,200 cost (CT=1200) would have an estimated 68.4% probability of being considered a specialty medication by this model. The above equations are for illustration only.

In some embodiments, model validation can be carried out using split sample methodology or bootstrap-based optimism correction and performance can be evaluated based on the area under the receiver operating characteristic curve (AUC). The cutoff for the binary SI rating can be established by a combination of performance analytics, including the AUC, and expert review by a clinical content team.

In some embodiments, the usefulness of this model depends on the predictive quality of the SI-PS. The variables considered in calculating the SI-PS may be updated in response to new data (e.g., indication-related variables or limited drug distribution status), regression diagnostics, or model performance. When the model selection is repeated, predictor variables that did not appear to have a substantial impact on SI-PS in initial modeling may be found to have an impact in later modeling that includes a broader scope of medications or variables. Conversely, predictor variables that at one time point appear to have a substantial impact on SI-PS may later appear to have very little impact and could be removed.

Features of the model itself could also be adapted and updated over time depending on findings in regression modeling, diagnostics, or performance. The initial modeling may be performed as described above, but the system could consider more sophisticated models as the data sample grows (e.g., mixed effects modeling to account for some hierarchical characteristics of the data, incorporation of more algorithmic model training methods such as iterative optimization and hyperparameter tuning, and others), if these models substantially improve performance.

In some embodiments, the SI-PS model is periodically recalculated to incorporate new data and revalidated. The SI-PS scores and SI values may change over time as the model is informed by a wider range of data points. As discussed above, the model may also be updated to include new parameters whose significance only becomes apparent with a larger sample, or to exclude previously included parameters whose predictive value was found to be previously overestimated. As such, the SI-PS model can be updated by tuning the model parameters and/or tuning the model variables.

In some examples, the system makes one prediction of SI-PS and SI for a pipeline drug that does not have complete information using the SI-PS model. Then the system makes a different prediction of SI-PS and SI for the same drug once more complete information is available using the same SI-PS model. In some examples, as more data of more drugs are added, e.g. as more pipeline drugs become approved drugs, the system can generate a more robust modeling or training dataset to be used for a periodic recalibration of the model.

In some embodiments, the SI-PS model is a machine learning model updated based on a plurality of candidate variables of additional medications. For each of the plurality of candidate variables, it is evaluated whether a correlation between the candidate variable and a specialty medication status predetermined for the additional medications is higher than the threshold, based on logistic regression with maximum likelihood estimation.

In some embodiments, the SI-PS model is a machine learning model updated based on additional candidate variables of existing medications. The model selection process 800 may be reinitiated including new candidate variables in the candidate variable pool. A new candidate variable may be included in the final model or not based on model selection criteria. If a new variable is included in the final model, other elements of the model such as the magnitude of other parameter estimates and the composition of the final model may change compared with prior models.

In some embodiments, the disclosed system has minimized the risks of poor model performance by: extensively researching the factors that are most important to formulary decision makers in establishing whether a medication should be considered a specialty medication; collecting the relevant data based on rules and logics set by broad and multidisciplinary experts; thoroughly evaluating the potential methodologic choices to create the SI-PS and SI models; and adapting the models and methodology based on any new data or user feedback.

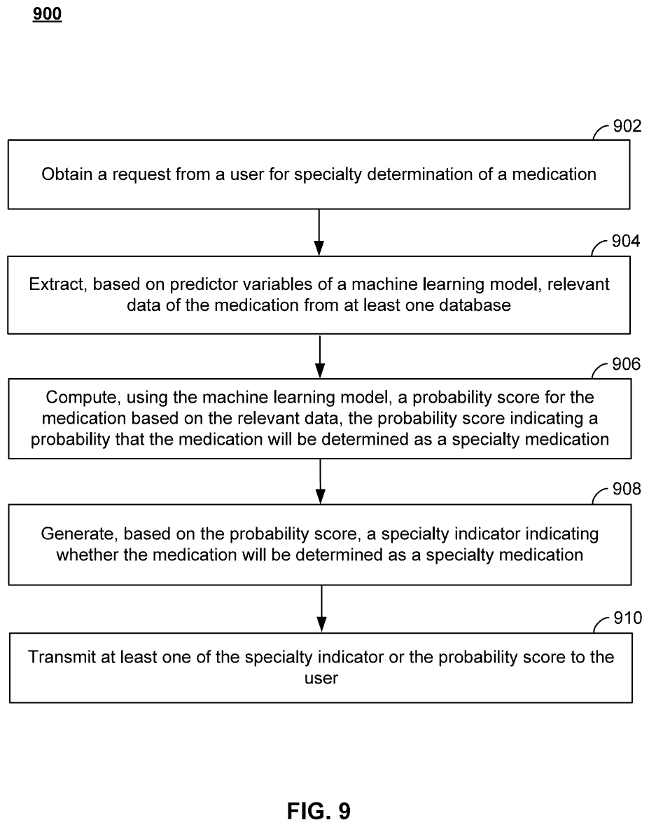

FIG. 9 is a flowchart illustrating an exemplary method 900 for automatically determining and indicating specialty medications, in accordance with some embodiments of the present teaching. In some embodiments, the method 900 can be carried out by one or more computing devices, such as the specialty indication computing device 102 and/or the cloud-based engine 121 of FIG. 1. Beginning at operation 902, a request is obtained from a user for specialty determination of a medication. At operation 904, relevant data of the medication is extracted from at least one database based on predictor variables of a machine learning model. At operation 906, a probability score is computed for the medication based on the relevant data, using the machine learning model, the probability score indicating a probability that the medication will be determined as a specialty medication. At operation 908, based on the probability score, a specialty indicator is generated to indicate whether the medication will be determined as a specialty medication. At operation 910, at least one of the specialty indicator or the probability score is transmitted to the user.

Although the methods described above are with reference to the illustrated flowcharts, it will be appreciated that many other ways of performing the acts associated with the methods can be used. For example, the order of some operations may be changed, and some of the operations described may be optional.

The methods and system described herein can be at least partially embodied in the form of computer-implemented processes and apparatus for practicing those processes. The disclosed methods may also be at least partially embodied in the form of tangible, non-transitory machine-readable storage media encoded with computer program code. For example, the steps of the methods can be embodied in hardware, in executable instructions executed by a processor (e.g., software), or a combination of the two. The media may include, for example, RAMs, ROMs, CD-ROMs, DVD-ROMs, BD-ROMs, hard disk drives, flash memories, or any other non-transitory machine-readable storage medium. When the computer program code is loaded into and executed by a computer, the computer becomes an apparatus for practicing the method. The methods may also be at least partially embodied in the form of a computer into which computer program code is loaded or executed, such that, the computer becomes a special purpose computer for practicing the methods. When implemented on a general-purpose processor, the computer program code segments configure the processor to create specific logic circuits. The methods may alternatively be at least partially embodied in application specific integrated circuits for performing the methods.

Each functional component described herein can be implemented in computer hardware, in program code, and/or in one or more computing systems executing such program code as is known in the art. As discussed above with respect to FIG. 2, such a computing system can include one or more processing units which execute processor-executable program code stored in a memory system. Similarly, each of the disclosed methods and other processes described herein can be executed using any suitable combination of hardware and software. Software program code embodying these processes can be stored by any non-transitory tangible medium, as discussed above with respect to FIG. 2.

The foregoing is provided for purposes of illustrating, explaining, and describing embodiments of these disclosures. Modifications and adaptations to these embodiments will be apparent to those skilled in the art and may be made without departing from the scope or spirit of these disclosures. Although the subject matter has been described in terms of exemplary embodiments, it is not limited thereto. Rather, the appended claims should be construed broadly, to include other variants and embodiments, which can be made by those skilled in the art.

What is claimed is:

1. A system, comprising:
a non-transitory memory having instructions stored thereon; and
at least one processor communicatively coupled to the non-transitory memory, and configured to read the instructions to:
obtain a request for specialty determination of a medication;
extract, based on predictor variables of a machine learning model, relevant data of the medication from at least one database;
compute, using the machine learning model, a probability score for the medication based on the relevant data, wherein the probability score indicates a probability that the medication will be determined as a specialty medication;
generate, based on the probability score, a specialty indicator indicating whether the medication will be determined as a specialty medication; and
transmit at least one of the specialty indicator or the probability score to a user.

2. The system of claim 1, wherein:
the relevant data comprises an adverse effect complexity code indicating a degree of adverse effect resulting from the medication; and
the adverse effect complexity code is one of: low, moderate or high, assigned based on an input of an expert and at least one predetermined rule.

3. The system of claim 1, wherein:
the relevant data comprises a monitoring parameters complexity code indicating a degree of monitoring needed for a patient taking the medication; and
the monitoring parameters complexity code is one of: low, moderate or high, assigned based on an input of an expert and at least one predetermined rule.

4. The system of claim 1, wherein:
the relevant data comprises a patient engagement critical to success code indicating a degree of patient engagement needed for a therapy based on the medication to be successful; and
the patient engagement critical to success code is one of: low, moderate or high, assigned based on an input of an expert and at least one predetermined rule.

5. The system of claim 1, wherein the at least one processor is configured to develop the machine learning model based on a plurality of candidate variables that comprise at least one measure of:
indication from medications;
medication types of the medications;
cost of therapy for the medications;
special storage conditions for the medications;
requirements for administration by health-care providers;
inclusion in a risk evaluation and mitigation program;
adverse effect complexity;
medication monitoring complexity; and
patient engagement complexity.

6. The system of claim 5, wherein the at least one processor is configured to develop the machine learning model based on:
for each of the plurality of candidate variables:
determining whether a parameter estimate P-value for the candidate variable and a specialty medication status predetermined for the medications is lower than a threshold, based on logistic regression with maximum likelihood estimation, wherein the specialty medication status is predetermined by an expert team;
in accordance with a determination that the P-value is lower than the threshold, incorporating the candidate variable as one of the predictor variables in the machine learning model; and
in accordance with a determination that the P-value is not lower than the threshold, excluding the candidate variable from the machine learning model.

7. The system of claim 6, wherein the at least one processor is configured to update the machine learning model based on:
obtaining additional medications; and
evaluating, for each of the plurality of candidate variables, whether a parameter estimate P-value for a relation of the candidate variable with specialty medication status predetermined for the additional medications is higher than the threshold, based on logistic regression with maximum likelihood estimation.

8. The system of claim 6, wherein the at least one processor is configured to update the machine learning model based on:
obtaining additional candidate variables;
evaluating, for each of the additional candidate variables, whether a parameter estimate P-value for a relation of the additional candidate variable with the specialty medication status predetermined for the medications is lower than the threshold, based on logistic regression with maximum likelihood estimation; and
updating the predictor variables in the machine learning model and their parameter estimates based on the evaluating.

9. The system of claim 1, wherein:
in accordance with a determination that at least one of the predictor variables has a missing value for the medication, an “unknown” value is assigned for the at least one predictor variables to the machine learning model for computing the probability score.

10. The system of claim 1, wherein the at least one processor is configured to:
transmit both the specialty indicator and the probability score to the user.

11. A computer-implementable method, comprising:
obtaining a request from a user for specialty determination of a medication;
extracting, based on predictor variables of a machine learning model, relevant data of the medication from at least one database;
computing, using the machine learning model, a probability score for the medication based on the relevant data, wherein the probability score indicates a probability that the medication will be determined as a specialty medication;
generating, based on the probability score, a specialty indicator indicating whether the medication will be determined as a specialty medication; and
transmitting at least one of the specialty indicator or the probability score to the user.

12. The computer-implementable method of claim 11, wherein the relevant data comprises:
an adverse effect complexity code indicating a degree of adverse effect resulting from the medication;
a monitoring parameters complexity code indicating a degree of monitoring needed for a patient taking the medication; and
a patient engagement critical to success code indicating a degree of patient engagement needed for a therapy based on the medication to be successful,
wherein each of the adverse effect complexity code, the monitoring parameters complexity code and the patient engagement critical to success code is one of: low, moderate or high, assigned based on an input of an expert and at least one predetermined rule.

13. The computer-implementable method of claim 11, further comprising developing the machine learning model based on a plurality of candidate variables that comprise at least one measure of:
indication from medications;
medication types of the medications;
cost of therapy for the medications;
special storage conditions for the medications;
requirements for administration by health-care providers;
inclusion in a risk evaluation and mitigation program;
adverse effect complexity;
medication monitoring complexity; and
patient engagement complexity.

14. The computer-implementable method of claim 13, wherein developing the machine learning model comprises:
for each of the plurality of candidate variables:
determining whether a parameter estimate P-value for the candidate variable and a specialty medication status predetermined for the medications is lower than a threshold, based on logistic regression with maximum likelihood estimation, wherein the specialty medication status is predetermined by an expert team;
in accordance with a determination that the P-value is lower than the threshold, incorporating the candidate variable as one of the predictor variables in the machine learning model; and
in accordance with a determination that the P-value is not lower than the threshold, excluding the candidate variable from the machine learning model.

15. The computer-implementable method of claim 14, further comprising updating the machine learning model based on:
obtaining additional medications; and
evaluating, for each of the plurality of candidate variables, whether a parameter estimate P-value for a relation of the candidate variable with specialty medication status predetermined for the additional medications is higher than the threshold, based on logistic regression with maximum likelihood estimation.

16. The computer-implementable method of claim 14, further comprising updating the machine learning model based on:
obtaining additional candidate variables;
evaluating, for each of the additional candidate variables, whether a parameter estimate P-value for a relation of the additional candidate variable with the specialty medication status predetermined for the medications is lower than the threshold, based on logistic regression with maximum likelihood estimation; and
updating the predictor variables in the machine learning model and their parameter estimates based on the evaluating.

17. The computer-implementable method of claim 11, wherein:
in accordance with a determination that at least one of the predictor variables has a missing value for the medication, an “unknown” value is assigned for the at least one predictor variables to the machine learning model for computing the probability score.

18. A non-transitory computer readable medium having instructions stored thereon, wherein the instructions, when executed by at least one processor, cause at least one device to perform operations comprising:
obtaining a request from a user for specialty determination of a medication;
extracting, based on predictor variables of a machine learning model, relevant data of the medication from at least one database;
computing, using the machine learning model, a probability score for the medication based on the relevant data, wherein the probability score indicates a probability that the medication will be determined as a specialty medication;
generating, based on the probability score, a specialty indicator indicating whether the medication will be determined as a specialty medication; and
transmitting at least one of the specialty indicator or the probability score to the user.

19. The non-transitory computer readable medium of claim 18, wherein the relevant data comprises:
an adverse effect complexity code indicating a degree of adverse effect resulting from the medication;
a monitoring parameters complexity code indicating a degree of monitoring needed for a patient taking the medication; and
a patient engagement critical to success code indicating a degree of patient engagement needed for a therapy based on the medication to be successful,
wherein each of the adverse effect complexity code, the monitoring parameters complexity code and the patient engagement critical to success code is one of: low, moderate or high, assigned based on an input of an expert and at least one predetermined rule.

20. The non-transitory computer readable medium of claim 18, wherein the instructions, when executed by the at least one processor, cause at least one device to further perform operations comprising developing the machine learning model based on a plurality of candidate variables that comprise at least one measure of:
indication from medications;
medication types of the medications;
cost of therapy for the medications;
special storage conditions for the medications;
requirements for administration by health-care providers;
inclusion in a risk evaluation and mitigation program;
adverse effect complexity;
medication monitoring complexity; and
patient engagement complexity.
